(19) United States
(12) Patent Application Publication
(10) Pub. No.: US 2011/0124064 A1
(43) Pub. Date: May 26, 2011

(54) ESTERIFICATION PROCESS OF PROSTAGLANDINS AND ANALOGUES THEREOF

(76) Inventors: Giancarlo Biffi, Milano (IT); Lazzaro Feliciani, Milano (IT); Alessandro D'Alfonso, Pavia (IT); Alessio Porta, Pavia (IT); Giuseppe Zanoni, Pavia (IT)

(21) Appl. No.: 12/996,374

(22) PCT Filed: May 8, 2009
(86) PCT No.: PCT/IB09/05536

§ 371 Date: Jan. 21, 2011

(30) Foreign Application Priority Data
Jun. 4, 2008 (IT) MI2008A001016

(51) Int. Cl. C12P 7/62

(52) U.S. Cl. 435/135

(57) ABSTRACT

The invention relates to a process for enzymatically catalyzed esterification of prostaglandins or analogues thereof.

### SUMMARY OF HE INVENTION

The present invention relates to a new process for preparing esters of prostaglandins and analogues thereof, in particular to a new enzymatically catalyzed esterification process.

### TECHNICAL BACKGROUND

Prostaglandins are a class of endogen molecules deriving from arachidonic acid through the action of prostaglandin synthetase and are provided with various biological activities.

Structurally, prostaglandins are formed by a ring (in the majority of cases a cyclopentane) and by two side chains, one of which with a terminal carboxyl group, said ring and chains being substitutable (usually by hydroxy or keto groups) and having possible unsaturations.

Compounds are known in which the terminal carboxyl group of the prostaglandins is esterified, such as derivatives of the prostaglandin PGF_{2α} latanoprost, travoprost and the analogue compound defined in the literature with the initials AL-12182 (see, for example, Chemistry Today, 2007, 25(1):58-60). These compounds have exhibited interesting antiglaucoma activity and activity against ocular hypertension.

Regardless of the synthetic approach used, the processes described for preparing esterified derivatives of prostaglandins and analogues thereof include a final step to esterify the carboxy group. In particular, type 2 nucleophilic substitution reactions of the corresponding carboxylate of the acid on a suitable electrophile are described.

The various known esterifications differ substantially by method of producing the nucleophile through deprotonation of the carboxyl group with bases such as Hunig's base, diisopropylethylamine (DIA), carbonates or hydroxides of alkaline and/or alkaline-earth metals. In particular, the use of diazabicycloundecene (DBU), K₂CO₃ and Cs₂CO₃ has been described.

The electrophilic component is instead usually a halide, such as bromide or iodide, an alkyl- or aryl-sulfonate, or a triflate.

However, prior art esterification processes have many drawbacks as they provide a product which must necessarily be purified and therefore require a reaction work-up with consequent formation even of large quantities of waste materials and consequent low yields, as well as the objective difficulties of treating unstable compounds such as prostaglandins and analogues thereof.

The simpler formation of the ester by reaction between the acid and an appropriate alcohol, a reaction which thus produces water as the only waste material, has never been seriously considered, as it has not yet been possible to make this esterification chemoselective. In fact, the hydroxy groups on the ring and on the side chain of prostaglandins and of analogues thereof compete with that of the alcohol used as esterifying agent and form with the carboxyl group intramolecular and intermolecular ester bonds, with the consequent formation of by-products that are difficult to eliminate.

Moreover, to obtain an esterification reaction by placing the acid and an appropriate alcohol in contact, it would be necessary to heat the reaction mixture, thus endangering the stability of the prostaglandins and analogues thereof which, as it is known, are thermolabile compounds.

There is therefore the need to provide an esterification process of prostaglandins and analogues thereof which is chemoselective, can be produced industrially and provides a final product of adequate purity, thus eliminating the need for further processing steps on the finished product and allowing a significant increase in reaction yields.

### DESCRIPTION OF THE INVENTION

It has now surprisingly been found that if the esterification reaction between the terminal carboxyl group of the prostaglandin (or an analogue thereof with a terminal carboxyl group) and a desired alcohol is carried out in the presence of a suitable enzymatic catalyst it is possible to prevent the production of by-products deriving from concomitant intramolecular and intermolecular esterification reactions.

Therefore, the invention relates to a process for preparing an ester of a prostaglandin or an analogue thereof with a terminal carboxyl group, which comprises reacting said prostaglandin or an analogue thereof with an alcohol, in the presence of an enzymatic catalyst.

According to the present invention, the term “prostaglandin” designates an endogenous prostaglandin or a synthetic prostaglandin, with a terminal carboxyl group.

According to the present invention, the term “prostaglandin analogue” designates a structural analogue of prostaglandins, of synthetic nature, with a terminal carboxyl group.

Preferred endogenous or synthetic prostaglandins are PGF_{2α} and analogues thereof.

Particularly preferred prostaglandin derivatives with an esterified carboxyl group are 13,14-dihydro-17-phenyl-18,19,20-trinor-PGF_{2α} (Latanoprost), 16-[3 -(trifluoromethyl)phenoxy]-17,18,19,20-tetranor-PGF_{2α} (Travoprost).

An esterified analogue of prostaglandins is for example the compound AL-12182 [(E)-isopropyl-7-(2-((E)-4-(3-chlorophenoxy)-3-hydroxybut-1-enyl)-4-hydroxytetrahydrofuran-3-yl)-hept-4-enoate)] (Chemistry Today, 2007, 25(1):58-60).

According to the present invention the alcohol as esterifying agent is selected according to the ester group to be formed. Preferred alcohols are, for example, linear or cyclic aliphatic alcohols, and aromatic alcohols, optionally substituted, such as (C₁-C₈)alkanols, (C₄-C₈)cycloalkanols, advantageously methanol, ethanol, isopropanol, butanols and cyclohexanol. A particularly preferred alcohol according to the present invention is isopropanol.

According to the present invention “enzymatic catalyst” is intended as a chemoselective enzyme capable of catalyzing the esterification reaction between carboxyl group and alcohol, at the same time preventing intramolecular and intermolecular esterification.

Examples of these catalysts are those in class EC3 according to the international enzyme nomenclature (www.chem.qmul.ac.uk/iubmb/enzyme/).

Preferred chemoselective enzymatic catalysts are selected from those in class EC3.1, in particular selected from lipases and esterases, of any origin.

Therefore lipases of animal origin can be used, such as porcine or eel lipases, or microbiological lipases, such as lipases from Candida antarctica, can be used. These catalysts are known to those skilled in the art and commercially available.

The enzymatic catalysts according to the present invention can be used in free, lyophilized, purified and partially purified form, or can be immobilized according to the various known techniques (CLEC, “cross-linked enzyme crystals”, CLEA, “cross-linked enzyme aggregates”, CSDE, “cross-linked spray dried enzymes” and CLE “cross-linked enzymes”), or can be trapped, for example in molecular cages, according to techniques known to those skilled in the art.

The esterification reaction can be carried out in a solvent. When possible and according to a preferred aspect of the invention, this alcohol, used in excess with respect to the acid, can act as solvent. Therefore, when alcohols such as methanol, ethanol, isopropanol, butanols or cyclohexanol are used, the esterification reaction can be carried out without other solvents.

If desired or required, a different solvent to the alcohol that participates in the reaction can be used. Suitable solvents are those inert to the esterification reaction and can be selected, for example, from cyclic or linear ethers and hydrocarbons, optionally halogenated.

The esterification reaction of the invention can be carried out at a temperature generally ranging from 0° C. to 50° C., advantageously below 40° C., preferably between room temperature and 35° C., for example around 30° C.

Advantageously, the temperature must never exceed the critical temperature for the stability of the enzymatic catalyst used and/or of the prostaglandin or analogues thereof used.

However, the use of enzymatic catalysts has the further advantage of being able to operate without the need to heat the reaction mixture, for example at room temperature or at a slightly higher temperature, thus without the risk of compromising the stability of the prostaglandin (or an analogue thereof) which, as mentioned above, is known to be a thermolabile compound. The fact that it is possible to operate without the need to heat the reaction mixture also represents a significant advantage from an industrial viewpoint, with consequent economic saving.

The reaction is completed in a period of time generally of less than 24 hours. Those skilled in the art can however monitor its progress through conventional techniques, such as TLC (Thin Layer Chromatography) or HPLC (High Pressure Liquid Chromatography).

At the end of the reaction the enzyme is normally removed by filtration and the solvent can be simply eliminated by distillation, for example at low pressure.

The desired ester is thus obtained directly in pure form, does not require any further purification treatment and the yield is almost quantitative.

Examples of reactions according to the invention are provided in the experimental section below, purely by way of example.

### EXPERIMENTAL SECTION

### Example 1

### Preparation of 13,14-dihydro-17-phenyl-18,19,20-trinor-PGF_{2α} acid isopropyl ester (latanoprost)

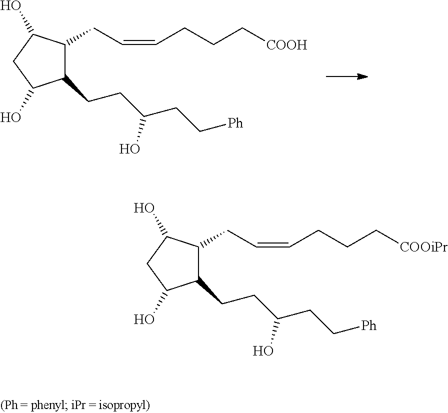

To a solution of acid (1 g, 2.56 mmols) in isopropyl alcohol (10 ml) the enzyme Lipase Novozym 435® (500 mg) is added. The mixture is kept at 30° C. under magnetic stirring (never exceeding 200 rpm). The reaction is controlled by TLC and terminates after 18 hours. The enzyme is simply filtered and recovered, the solvent is removed at low pressure to give a pure product in the form of pale yellow oil with a yield of 92%.

1H NMR (200 MHz, CDCl₃) δ: 7.2 (5H, m), 5.4 (2H, m), 5.0 (1H, m), 4.21 (1H, s), 3.9 (1H, s), 3.6 (1H, q), 2.6-2.9 (2H, m), 1.3-2.4 (18H, m) 1.2 (6H, d).

### Example 2

### Preparation of 13,14-dihydro-17-phenyl-18,19,20-trinor-PGF_{2α} , acid ethyl ester

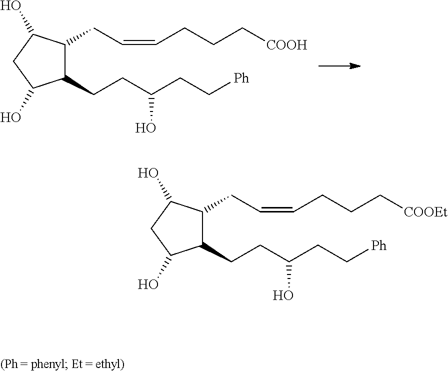

To a solution of acid (30 mg, 0.077 mmol) in absolute ethyl alcohol (350 μl) the enzyme Lipase Novozym 435 (15 mg) is added. The solution is kept at 30° C. under magnetic stirring (never exceeding 200 rpm). The reaction is controlled by TLC and terminates after 18 hours. The enzyme is simply filtered and recovered, the solvent is removed at low pressure to give a pure product in the form of transparent oil with a yield of 90%.

1H NMR (200 MHz, CDCl₃) δ: 7.2 (5H, m), 5.4 (2H, m), 4.2 (3H, m), 3.9 (1H, s), 3.6 (1H, q), 2.6-2.9 (2H, m), 1.3-2.4 (18H, m) 1.2 (3H, t).

### Example 3

### Preparation of 13,14-dihydro-17-phenyl-18,19,20-trinor-PGF_{2α} acid methyl ester

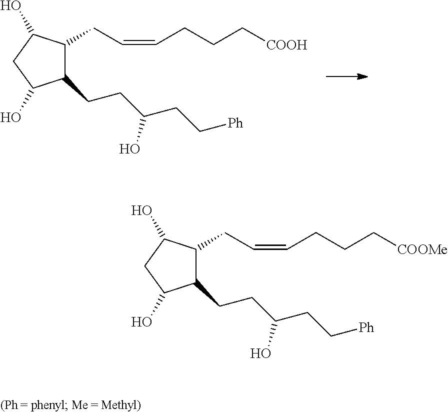

To a solution of acid (25 mg, 0.065 mmols) in ethyl methyl ether (6 ml) methanol (125 μl) and the enzyme Lipase Novozym 435 (16 mg) is added. The solution is kept at 30° C. under magnetic stirring never exceeding 200 rpm. The end of the reaction takes place after 5 hours and is controlled through TLC. The enzyme is filtered and recovered, the solution is then concentrated at low pressure. The product is in the form of colourless oil and a yield of 95% is obtained.

1H NMR (200 MHz, CDCl₃) δ: 7.2 (5H, m), 5.4 (2H, m), 4.21 (1H, s), 3.9 (1H, s), 3.6 (4H, m), 2.6-2.9 (2H, m), 1.1-2.4 (18H, m).

1. A process for preparing an ester of a prostaglandin or an analogue thereof with a terminal carboxyl group, which comprises reacting said prostaglandin or an analogue thereof with an alcohol, in the presence of an enzymatic catalyst.

2. The process according to claim 1, characterized in that said prostaglandin is an endogenous or synthetic prostaglandin, or a synthetic analogue thereof.

3. The process according to claim 1, characterized in that said prostaglandin or analogue thereof is selected from PGF_{2α} and analogues thereof.

4. The process according to claim 1, characterized in that said ester of a prostaglandin or of an analogue thereof is selected from 13,14-dihydro-17-phenyl-18,19,20-trinor-PGF_{2α} isopropyl ester (Latanoprost), 16-[3-(trifluoromethyl)phenoxy]-17,18,19,20-tetranor-PGF_{2α} isopropyl ester (Travoprost).

5. The process according to claim 1, characterized in that said ester of a prostaglandin or of an analogue thereof is the compound (E)-isopropyl-7-(2-((E)-4-(3-chlorophenoxy)-3-hydroxybut-1-enyl)-4-hydroxytetrahydrofuran-3-yl)-hept-4-enoate).

6. The process according to any one of claims 1 to 2, characterized in that said alcohol is selected from linear or cyclic aliphatic alcohols, optionally substituted.

7. The process according to claim 6, characterized in that said alcohol is selected from methanol, ethanol, isopropanol, butanols and cyclohexanol.

8. The process according to any one of claims 1 to 7, characterized in that said enzymatic catalyst is selected from the enzymes in class EC3.1.

9. The process according to claim 8, characterized in that said enzymatic catalyst is selected from lipases and esterases.

10. The process according to any one of claims 1 to 9, characterized in that the reaction is carried out at a temperature below 40° C.

11. The process according to claim 10, characterized in that the reaction is carried out at a temperature of around 30° C.

12. The process according to claim 7, characterized in that the reaction is carried out without other solvents.
